(19) United States
(12) Patent Application Publication
(10) Pub. No.: US 2025/0378425 A1
(43) Pub. Date: Dec. 11, 2025

(54) SYSTEM AND METHOD FOR TESTING COMMERICAL DRIVERS

(71) Applicant: Evolution Trust Us, Sarasota, FL (US)

(72) Inventor: Anthony Elwood, Bradenton, FL (US)

(73) Assignee: Evolution Trust Us, Sarasota, FL (US)

(21) Appl. No.: 19/235,551

(22) Filed: Jun. 11, 2025

Related U.S. Application Data
(60) Provisional application No. 63/658,620, filed on Jun. 11, 2024.

(51) Int. Cl. G06Q 10/1093; G06Q 10/02; G06Q 50/26; G16H 10/40

(52) U.S. Cl. CPC G06Q 10/1093; G06Q 10/02; G06Q 50/265; G16H 10/40

(57) ABSTRACT

Systems and methods are provided for streamlining the mandatory drug testing process for commercial drivers. The system includes a central database on a secure networked server provided in communications with several user applications. Drivers and/or their employers can register on the system, with registration data stored on a server database. Drug testing sites can also register with the system. The system can alert drivers to schedule testing through a driver application on a smartphone or tablet. The driver can schedule a test with a registered testing site and select a convenient site based on GPS data provided by their app to the system. Test results can be provided to appropriate government agencies, the driver, and the driver's employer. The driver app can be configured to schedule any required treatment with registered counselors, and counselors can report status of treatment to the driver, employer, and appropriate agencies.

### CROSS-REFERENCE TO RELATED APPLICATIONS

This application claims priority to U.S. provisional application No. 63/658,620 filed on Jun. 11, 2024.

### FIELD OF THE DISCLOSURE

The present disclosure relates to systems and methods for automating random commercial driver selection for mandatory drug test selection, providing efficient on-route drug test collection procedures, and securely and selectively reporting results to human resource (HR) personnel, Safety Managers, MIS, and the DOT clearinghouse to ensure conformance with all relevant privacy laws including HIPAA.

### BACKGROUND INFORMATION

Commercial motor vehicles (CMVs), such as 18-wheel trucks, play an important role in our national commerce, as it provides transport of enormous quantities of goods throughout our nation. These trucks require special driving skills, as they are very large and can be extremely heavy, so that operating them safely on the public roads is critical. Also, they are constantly on the road, accumulating many miles, and are generally mixed in traffic with smaller, less sturdy noncommercial vehicles. Accordingly, a special commercial driver's license (CDL) is required to legally drive CMVs.

The US Department of Transportation (DOT) administers commercial driving regulations to ensure safety on the roads. As part of this, it sets the rules for drug and alcohol testing procedures and reporting requirements of CDL holders. In addition to skill requirements needed to obtain a CDL, commercial drivers may be tested for drug or alcohol for the following reasons: Pre-employment, random tests, follow up testing if a driver has a positive test result, return to duty, and post-accident. For commercial drivers, a blood alcohol concentration (BAC) (determined via a standardized breath test) greater than 0.04 is a positive result. A blood alcohol concentration of 0.02-0.04 requires that the driver is removed from safety-sensitive position for at least 24 hours.

Title 49 of the Code of Federal Regulations (49 CFR) governs transportation-related rules, including the rules and regulations for testing commercial drivers and reporting results of drug and alcohol tests. Mandatory drug testing as defined under 49 CFR 40.437(b)(2) section 382.107 of the Intrastate and Interstate Commerce CDL requirement, is currently a manual process that requires commercial drivers to provide urine specimens when their name is chosen at random. Companies that have more than one CDL driver are required to place their drivers in a consortium for random testing purposes. Finding consortiums can be a daunting task for some companies. Once a consortium is located, the company has to manually add driver information, ensure that randomly selected tests are completed, and report the results to the DOT's Management Information System (MIS) and the Federal Motor Carrier Safety Administration's (FMCSA) Drug and Alcohol Clearinghouse (DAC) system. The DAC is a secure online database that identifies operators of CMVs and centralizes records of their alcohol and controlled substance testing, including test results that violate FMCSA's Drug and Alcohol Testing regulations.

Beginning in November 2024, a commercial driver having a “prohibited” status in FMCSA's Drug and Alcohol Clearinghouse system due to a drug and alcohol program violation will lose or be denied a commercial driver's license (CDL) or commercial learner's permit (CLP). As part of new Federal regulations, FMCSA is partnering with State Driver Licensing Agencies (SDLAs) so that drivers with a “prohibited” Clearinghouse status will lose or be denied their state-issued commercial driving privileges. This adds a layer of safety, making it more difficult for drivers to conceal their drug and alcohol program violations from current or prospective employers and evade the commercial motor vehicle driving prohibition. It also supports FMCSA's goal of ensuring that only qualified drivers are eligible to receive and retain a commercial license, thereby making roadways safer for motor carriers and the traveling public.

On May 2, 2023 The Department of Transportation announced the intention to allow oral fluid specimen (saliva) testing for commercial drivers, instead of the current urine and breath sampling standards. This method of specimen testing represents an improvement for the drug testing aspects of the transportation industry. Historically, drug test specimens were based on collected urine samples, which allowed drivers three hours to produce a specimen. By allowing oral collections, commercial drivers no longer face the obstacles of ‘shy bladder,’ which may extend a drug test to up to three hours.

The upcoming DOT 5-panel saliva drug test protocol covers the same drugs tested in the current DOT urine 5-panel test array. Oral testing will detect five controlled substances currently in the body or recently used within the last 48 hours. These substances tested for are THC, opiates, PCP, cocaine, and amphetamines.

There are many advantages to the recently-announced oral saliva testing. For example, it is less invasive than urine testing, and same-sex observers are not required for sample collection. It is also more time efficient, as oral saliva can be collected in less than five minutes instead of the up to three hours allowed to collect a urine sample. Also, private bathrooms are no longer required (as is the case for urine testing), so the individual being tested can be observed for the entire time of the sample collection. And, oral fluid/saliva is harder to adulterate than collected urine samples. Further, transgender and nonbinary individuals that now require a DOT Direct Observed collection can proceed with oral saliva collection without requiring a same-gender collector. Finally, saliva test results are available much quicker, with negative results available in 24-36 hours and positive results in 72-96 hours.

When either a drug test or a BAC test is positive, the driver must seek the resources of a Substance Abuse Professional (SAP) to maintain their CDL. At the same time, a Designated Employer Representative (DER) for that driver, typically someone associated with the driver's employer, will be alerted to a positive drug or alcohol result, so they may immediately remove the driver from the safety sensitive position. At the same time, the Designated Employer Representative (DER) for that driver will be alerted to a positive drug or alcohol result, so they may immediately remove the driver from the safety sensitive position.

It is important to distinguish between a drug test result and a drug test report for commercial drivers. A drug test result is the conclusion of a drug or alcohol test, which is either positive or negative. On the other hand, a drug test report provides the specific details about the donor, company name requesting the drug or alcohol test, the types of drugs tested for, allowable cutoff levels, and the levels detected in the driver's body, together with the Medical Review Officer's (MRO) signature, and the result. A positive drug test result, reported to a DER, requires that the commercial driver removed from duty for a period of time. Under current regulations, the DER should only receive notice of a positive test result, without any details about the specific drug in the driver's system that led to the positive result.

If a commercial driver who has a positive drug or alcohol test result is pulled from the road, he or she can regain their commercial driving privileges and be allowed to drive commercial vehicles if they successfully complete a treatment program under the guidance of a registered Substance Abuse Professionals (SAP) The SAP will provide an appropriate course of treatment and/or counseling based on the nature of the failed test, evaluate the driver and set a schedule for follow-up testing and Return to Duty Testing.

The administration of commercial driving privileges, which includes maintaining a database of commercial drivers, administering drug and alcohol tests to ensure safety, monitoring driver status, and coordinating with SAPs to reinstate drivers having a positive test result if they successfully complete their treatment program, is a complex and involved process with many facets. Accordingly, there are several inefficiencies and issues with the current systems that are in place to ensure that commercial drivers are safe to operate commercial vehicles on the nation's roadways.

For example, there are significant financial and time costs for commercial drivers to comply with the government's mandatory testing programs. A CDL driver will typically lose about 120 miles in a three hour window to complete mandatory drug testing under the current system. This includes the necessity to drive off-route to report to a test collection site, and possible scheduling delays encountered at such sites. There are about 3 million commercial drivers in the USA. Half of these drivers must be tested to meet DOT compliance requirements. Thus, about 1.75 Million drivers will lose a total of about 210 million miles in freight delivery per year due to testing requirements.

An average driver makes about $2,000 per week, which is about $50/hour. It takes about three hours to complete drug testing off route. At $50/hour, it can cost a company about $150 per driver to complete drug testing. The total nationwide annual revenue lost due to drug testing compliance is about 250 million dollars, which is a significant expense across the commercial transport industry. Although the upcoming saliva-based testing can reduce the time required for such testing to a degree, much time and many miles are wasted when commercial drivers are required to detour from their assigned routes to a designated test collection site.

Another significant issue that arises with the current driver testing and treatment procedures relates to privacy issues. HR and Safety Managers responsible for mandatory drug testing, as defined under 49 CFR 40.437(b)(2) section 382.107 of the Intrastate and Interstate commerce CDL requirement, have a rigorous task of manually meeting compliance. Currently, HR and Safety Managers must:
Enroll all CDL divers in a randomization pool;
Select drivers from the random pool;
Track testing to ensure 50% of their drivers were randomly selected;
Schedule the driver to complete the test off route;
Ensure all drivers randomly chosen have completed the test; and
Immediately upload results to the Management Information System of the Federal Motor Carrier Safety Administration (FMCSA).

As noted earlier, the Drug and Alcohol Clearinghouse (DAC) system is a secure online database that identifies operators of CMVs and centralizes records of their alcohol and controlled substance testing. Although the Clearinghouse is secure, it is not encrypted and any person who has a password can access the database and obtain drug test reports that specify the details of any test results of commercial drivers contained in the database.

Drug and alcohol abuse by a commercial driver are considered an illness that can be treated, and thus information about such abuse (which is identified in drug test reports contained in the Clearinghouse database) represents medical information that is subject to privacy laws under the Health Insurance Portability and Accountability Act of 1996 (HIPAA), which generally prohibits healthcare providers and businesses called covered entities from disclosing protected information to anyone other than a patient and the patient's authorized representatives without their consent. The Americans with Disabilities Act (ADA) also protects workers from repercussions by their employer if they have been successfully rehabilitated and are no longer engaged in the illegal use of drugs; if they are currently participating in a rehabilitation program and are no longer engaging in the illegal use of drugs; and if they are regarded, erroneously, as illegally using drugs.

To conform with current privacy laws and regulations, employers of commercial drivers should have access only to drug test results, which (in the case of a reported positive result) is sufficient to remove a driver from their driving responsibilities in compliance with Federal regulations. However, in the current implementation, drug test reports and results are provided to DER and HR representatives by email, mail, and other platforms (ex: DISA, Red Arrow, etc.). All users having access to these systems may get both results and reports, and the data is not encrypted. Drug test reports, which contain all of the details of a positive test, including the type of drug(s) detected and their levels. Although such access to drug test reports by employers can violate HIPAA and ADA laws, it nevertheless occurs in the real world. With such unauthorized information, employers may decide to permanently terminate employment of CDL drivers, rather than allowing them to participate in and complete an approved counseling program administered by a SAP to regain their driving privileges and keep their jobs.

Additionally, the lack of proper security and encryption of drug test report records allows for the potential of DER and HR personnel to leak results/reports to other employees who would not otherwise have access to them. These employees may feel dissatisfaction with the company by perceiving the continued employment of a driver who tested positive as unfair, not knowing that the driver is complying with follow up treatment and testing as prescribed by the SAP. In summary, the real-world lack of sufficient security and privacy with respect to drug test reports can lead to unfair treatment and improper termination of drivers, potential violation of HIPAA and/or ADA laws, and employee issues within the company.

Loss of a portion of the commercial driver workforce due to issues with drug testing, lack of confidentiality, and difficulties in obtaining, coordinating, and reporting of treatment and reinstatement procedures represents a significant problem that spans the entire commercial transportation industry.

For example, more than 56,000 drug and alcohol violations were recorded in 2023 in a database intended to track truck drivers' compliance history and prevent them from job-hopping in the event of a failed drug test. The number of reported driver violations rose by roughly 10,000 over the final two months of 2020, the first full year of operation for the Federal Motor Carrier Safety Administration's Drug and Alcohol Clearinghouse. Of the roughly 45,000 commercial drivers who lost their job due to positive drug test results that year, 34,000 have not yet completed a return-to-work program to regain their CDL privileges. This large percentage indicates that many drivers may be leaving the industry permanently. This trend may severely impact the capacity of our country's commercial transportation network.

Accordingly, there is a need for systems and methods for improved tracking and monitoring commercial driver records, including ensuring compliance with all Federal reporting and testing requirements and ensuring security and privacy of drug test reports to comply with HIPAA and ADA laws, as well as simplifying and streamlining such reporting procedures to reduce the burden on employers of commercial drivers. There is also a need for simplifying and streamlining treatment and reinstatement protocols for drivers who test positive, to encourage them to regain their driving privileges and maintain an effective level of available drivers for the commercial transport industry.

### SUMMARY OF THE DISCLOSURE

The present disclosure describes novel systems and methods that provide a streamlined procedure for commercial drivers to undergo mandatory drug testing that reduces lost time and miles that occur under current protocols. The disclosed systems and methods also improved tracking and monitoring of commercial driver records that ensures compliance with all Federal reporting and testing requirements. Communication of confidential information related to drug testing results is also more reliable and secure through the use of particular communication channels that ensure compliance with HIPAA and ADA laws based on imposing stricter security and privacy of drug test reports, where such security and compliance is not generally present in current manual reporting procedures. Further embodiments of the disclosed systems and methods can also facilitate and track appropriate treatment and therapy for drivers who test positive, thereby helping to keep more commercial drivers on the road who are in conformance with Federal drug testing requirements.

Embodiments of the disclosure are based on a platform that includes a computer server arrangement having appropriate hardware and software configured to enable secure electronic communication between various types of users and databases located on the server. The interface between users and the system can be provided by various means including, e.g., smartphone and portable tablet apps, and software programs running on networked computers. Encryption of all such networked communication is preferably provided to ensure security of data being communicated.

The core of the disclosed system is a Central Administration (CA) facility or system, which includes a networked computer server housing a secure CA database stored in nonvolatile computer memory, and communications capabilities that provide networked communication of particular information to particular users of the system based on their position and status. Such networked communications can be wired and/or wireless, where wireless communications can be used to securely send and receive certain information to particular users via one or more system applications that can be accessed via smartphones, tablets, or the like.

The server also comprises server software stored in nonvolatile computer memory that is configured to process received data, access and modify the CA database, perform various functions as described herein, and to communicate information with system apps via the communications capabilities, which can include both wired and wireless communication pathways.

Individual commercial drivers can be registered in the system directly or through their employers. Such registration can be performed using applications provided to employers and drivers, which can provide secure enrollment and registration in the system. Enrollment information can include, e.g., a driver's name, personal data such as home address, date of birth, etc., and their CDL number. Drivers can access the system via a smartphone app or other communication device that also has location detection capabilities (e.g., GPS functions), and registration information can be communicated and stored securely on the CA database.

The CA system can be used to coordinate and comply with Federal requirements for the randomized drug testing program, whereby all drivers registered with the CA database can be associated with the Federally required drug testing randomization program and thus become part of the randomized testing pool with minimal effort on their part. Once a driver is selected to provide a random drug screen, they will be notified via the system app of their obligation to submit to a screening test. The employer of the driver (if they're not independent) can also receive a report of their selection.

Approved drug test collection sites can also register with the disclosed system. When a driver is required to take a drug screening test, the location detection function of the driver's device (e.g. smartphone) can be used to provide the current location of the selected driver. Upon notification of a driver's test requirement, the CA system can provide the locations of registered test collection sites that are located conveniently near the driver or along the route. The system app can allow a driver to select a particular test collection site and a time or time interval when they will be present for testing at the site. Such information can then be communicated by the system to the test collection site to improve their scheduling and minimize nonproductive off-route time for the driver being tested.

In certain embodiments, test collection sites can improve their scheduling and supply efficiencies through the real-time networked scheduling capabilities. Optionally, verification of driver credentials, security of provided samples, and tracking of test sample shipments to testing labs can be easily performed by testing sites using the system and an associated app that is configured for use by the sites to perform these functions. The registered test collection sites can be set up to comply with all DOT Specimen Collection Procedures Guidelines, as specified in 49 CFR 40, for Transportation Workplace Drug Testing Programs.

Certified testing labs can also be registered with the systems, and prepare drug test results and reports based on the samples received from the testing sites. The results can then be communicated securely to an authorized Medical Review Officer (MRO) independently or through the described system. The MRO can then review and record the test results and reports, which can then be communicated securely to the DOT Clearinghouse as required under current regulations. This information can also be sent by the MRO to the CA database to be stored securely therein, optionally in an encrypted format with controlled access to certain portions of such drug test information being made available to employers and drivers, respectively, in accordance with HIPAA laws and other applicable regulations.

The system can also be configured to send drug test reports directly to employers of the tested drivers that only indicate a positive or negative result, which alerts the employer if the driver is not qualified to continue working based on a positive test result. The full test report (containing details of the specimen analysis) can be sent securely and directly to the driver as part of the driver's private medical records, thus maintaining confidentiality of the driver's medical information and ensuring compliance with HIPAA and ADA laws.

In operation, a system app can allow commercial drivers to register with the disclosed system and create a user profile, enter their credit card information, and digitally sign/authorize consent forms for electronic data, any required disclosures, and HIPAA agreements. Such registration can also be performed by employers of drivers, allowing company-wide management of driver certifications and status.

Further embodiments of the disclosure can also provide a system and platform for facilitating treatment and/or therapy protocols for drivers who have tested positive for drugs, in order to motivate and help them to receive such help and regain their driving privileges. For example, after a driver tests positive and is restricted from driving, the system/platform, via the end user system app, can provide the driver with a list of substance abuse professionals (SAPs) registered with the system who are in close proximity to the driver's home address or any other provided location. Once the driver chooses a SAP, and with the driver's consent, the drug test report can be encrypted by the CA database system and sent to the Substance Abuse Professional for further evaluation. The driver can then connect with the selected SAP directly, and comply with any follow-up testing and return-to-duty testing requirements as prescribed by the SAP.

SAPs may evaluate drivers remotely or in person. The system can provide an appropriate interface (e.g., visual teleconference connections) or direct communication information (e.g. a web link to allow the driver and SAP to teleconference, etc.) to allow the SAP to meet the criteria for conducting remote evaluations, and to report the progress and completion of such evaluations and associated treatment to the driver, the driver's employer, and to authorities who track and confirm the completion of these requirements.

For example, the platform can also provide an encrypted web interface for SAPs to schedule follow up testing for the driver, and provide resources to the driver to schedule any further tests and receive the results, to ensure the driver completes the follow up testing schedule as ordered by the SAP. The system and platform described herein can also be configured to receive and store a prescribed testing schedule provided by the SAP for that driver, so that the driver can be notified, e.g., on the day that testing is required, and be provided with the test collection site location.

Results of any follow up tests or return to work tests can be reported automatically by the system to the driver's employer to meet Federal compliance requirements. Both drug test information and any required treatment/counseling procedures can also be uploaded to the Management Information System (MIS) to comply with government protocols. This post-positive result feature of the disclosed system makes it easier for a driver to obtain services from an SAP after testing positive, and to successfully and conveniently engage in appropriate therapy and/or treatment. It also simplifies the scheduling and reporting activities of an SAP registered with the system so they can focus more on the actual interactions with the driver.

### BRIEF DESCRIPTION OF THE DRAWINGS

Further objects, features and advantages of the disclosure will become apparent from the following detailed description taken in conjunction with the accompanying figures showing illustrative examples, results and/or features of the exemplary embodiments of the present disclosure, in which:

FIG. 1 is a flowchart showing the main components and communication lines for a system that provides more efficient and secure communication, scheduling, access to, and secure transmittal of results for required drug testing activities for commercial drivers; and

FIG. 2 is a flowchart showing communication lines for both commercial drivers and drug testing sites within the improved drug testing system for commercial drivers described herein.

The various embodiments of the disclosure are described herein with reference to the figures, where like reference numbers indicate identical or functionally similar elements. Further features and advantages of the disclosure as well as the structure and operation of various embodiments of the present disclosure are described in detail below with reference to the accompanying drawings. To the extent that the present disclosure does reference the figures, it is done so in connection with the illustrative embodiments and is not limited by the particular embodiments illustrated in the figures. It is intended that changes and modifications can be made to the described embodiments without departing from the true scope and spirit of the present disclosure.

### DETAILED DESCRIPTION OF EXEMPLARY EMBODIMENTS

Embodiments of the present disclosure provide novel systems and methods for improved tracking and monitoring of commercial driver records that ensures compliance with all Federal reporting and testing requirements. The disclosed systems and methods provide a streamlined procedure for drivers to undergo mandatory drug testing to reduce lost time and miles that occur under the current system. The disclosed systems and methods also provide improved security and restrict communication channels appropriately to ensure compliance with HIPAA and ADA laws based on imposing stricter security and privacy of drug test reports. Reporting procedures are also simplified and streamlined to reduce the burden on employers of commercial drivers. Finally, the disclosed systems and methods can also provide a simpler process for drivers who test positive to receive appropriate treatment and therapy, thus motivating them to regain their driving privileges and livelihood and maintaining a larger pool of approved commercial drivers in view of Federal drug testing requirements.

Embodiments of the disclosure are based on a platform that includes a networked computer server hosting a secure CA database, and configured with communications capabilities that provides networked communication of particular information to particular users of the system based on their position and status. The interface between users and the system can be provided by various means including, e.g., smartphone and portable tablet applications (e.g. user apps), and software programs running on networked computers. Encryption of all such networked communication is preferably provided to ensure security of data being communicated.

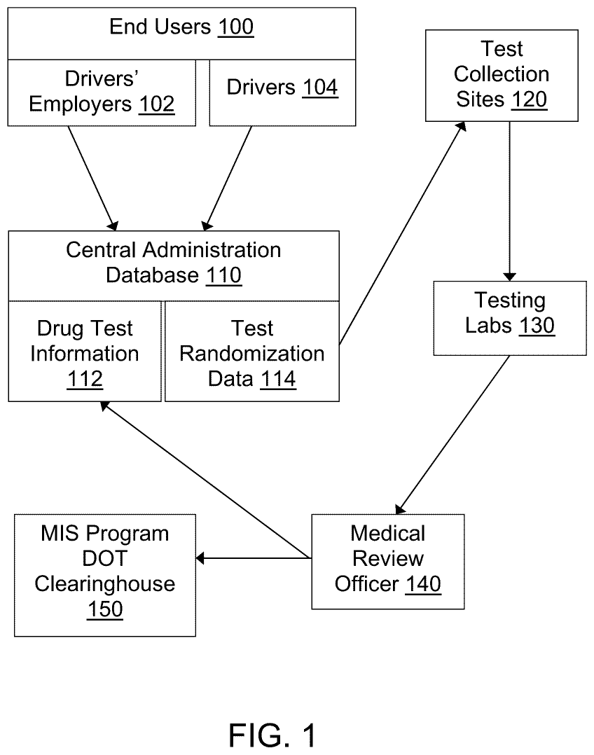

A schematic diagram of the primary components and communications paths of the disclosed system are provided in FIG. 1. A Central Administration (CA) system 110, which includes a server hosting a secure CA database 110 and communications capabilities, is a core component of the system that can be directly accessed only by approved administrators.

End users 100 of the system include individual commercial drivers having a CDL 104, drivers having a Driver Training Certificate (DTC) 104, and companies 102 that employ and/or monitor commercial drivers, including the HR and Safety Manager personnel of such companies 102. The End User portal will allow HR and Safety Managers of companies to enroll drivers 104 in the government randomization testing program. Individual drivers 104 can also enroll in the system. Enrollment can be done via a registration form provided through an app and/or computer program that is in communication with the Central Administration database 110, and the randomization program data 114 can be stored in the CA database 110. Enrollment information can include, e.g., a driver's name, personal data such as home address, date of birth, etc., and their CDL number. Preferably, end users 100 can access the system via a smartphone app or other communication device that also has location detection capabilities (e.g., GPS functions). Smartphones owned by most drivers 104 already have such communication and location functions, so an end user app on such a device can be used to achieve the required functionalities needed by the end user.

The CA system can be used to coordinate and comply with Federal requirements for the randomized drug testing program. Current procedures include manually entering drivers into a random pool. Most companies achieve this by pulling an active list of drivers every quarter, submitting the list to their Drug and Alcohol consultant, and waiting to receive a list of drivers who need to be tested. Then the companies must track down their drivers and ensure each driver completes random testing. If a driver selected for random is not available, alternates must be chosen.

The present system streamlines this process significantly. A company's active driver roster can be stored securely in the CA database 110 directly through the platform, or alternatively the roster can be pulled by the present system and platform via API integration with a company's fleet management software. This eliminates the need for company personnel to manually pull of rosters. For example, all drivers 104 registered with the CA database 110 will be associated with the Federally required drug testing randomization program. Non-contractual truck drivers 104 enrolled in the present system can be directly assigned to a consortium and thus become part of the randomized testing pool with minimal effort on their part.

Through the randomization program, certain drivers 104 will be randomly selected from the randomization program data 114 to complete an oral saliva drug test collection. Once a driver 104 is selected to provide a random drug screen, the driver 104 will be notified via the system app of their obligation to submit to a screening test. HR and Safety Managers affiliated with the driver 104 will also receive a report of their selection.

A database of approved drug test collection sites can be included in the CA database, and the location detection of the end user's device (e.g. smartphone) can be used to provide the current location of the selected driver 104. Immediately upon notification, the driver 104 will have the option to select a test collection site 120 near their location. Optionally, a driver's current route can be provided by the driver 104, and the CA system can then provide the locations of test collection sites 120 that are located conveniently along the route. Operating hours of the test collection sites 120 and their current testing status/availability can also be provided to the driver 104, as well as (optionally) available amenities at the test collection sites 120 (e.g., gas, showers, food, etc.). Additionally, the app can allow a driver 104 to select a particular test collection site 120 and a time or time interval when they will be present for testing at the site. Such information can then be communicated by the system to the test collection site 120 to improve scheduling at the test collection site 120.

If a test collection site 120 is affiliated with the disclosed system (e.g., not an independent site), the system can be configured to allow affiliated sites to track their stock of supplies needed for testing, and notify the CA administrative users when it's time to order additional supplies for that test collection site 120. Test collection sites 120 can also be provided with step by step instructions for obtaining, labeling, and shipping the drug test specimens. API interfaces can optionally be used to track specimen shipments via the shipper's own database, thereby facilitating tracking updates of specimens between the test collection sites 120 and the testing labs 130 within the disclosed system and platform.

Through this part of the system that selects drivers 104 registered with the system for the required random testing, notifies selected drivers 104 that they must take a screening test, determines the driver's current location and/or upcoming route, and provides convenient test collection site options together with the ability to select a test collection site 120 and testing time, the required testing procedure can be greatly streamlined to minimize the time that a driver 104 is taken off route to undergo the screening procedure. Also, by providing a communication interface that coordinates the driver's participation with the test collection site's schedule, the disclosed system can improve the efficiency of the test collection site scheduling and reduce any potential delays at the site.

Test collection sites 120 registered with the disclosed system can be located at rest stops or close to major highways and thoroughfares. Their locations and testing schedules/operating hours can be provided by the site personnel to the CA database. These test collection sites 120 will be set up to comply with all DOT Specimen Collection Procedures Guidelines, as specified in 49 CFR 40, for Transportation Workplace Drug Testing Programs. If necessary, the system (through its administrative users) can provide step by step instructions for the test collection site personnel, and communications can be provided whereby any questions or problems that may arise at the test collection site 120 can be addressed by the system administrators. Such communication between the CA personnel and test collection site personnel can be via the network, or telephone numbers can be provided to facilitate rapid communication between them.

Testing procedures at the test collection sites 120 can follow normal protocols, which includes obtaining scanning and verifying the driver's government issued ID, such as their CDL, obtaining a specimen from the driver and labeling it with a Chain of Custody (CoC) barcode to ensure donor/specimen continuity, and packaging the obtained and labeled specimens for pickup by a certified carrier such as FedEx and transport to an authorized testing lab 130.

The testing lab 130 then processes the specimens, prepares a test report for each specimen, and securely communicates the results to an authorized Medical Review Officer (MRO) 140. The testing labs 130 may be independent, or they may be registered with the Central Administration database 110 and optionally use the communication protocols of the present system to report test results and reports to the MRO 140.

The MRO 140 can then review and record the test results and reports, which can then be communicated securely to the DOT Clearinghouse 150 as required under current regulations. The test results and reports (collectively, drug test information 112) can also be sent by the MRO 140 to the CA database 110 to be stored securely therein, optionally in an encrypted format with controlled access to certain portions of such drug test information 112 being made available to employers 102 and drivers 104, respectively.

The system can be configured to send drug test reports directly to employers 102 of the tested drivers 104. This conforms to Federal regulations, as the employers 102 can then remove drivers 104 who test positive from active driving duties. An important feature of the system is that only the test reports are communicated to the employers 102 (e.g., a positive or negative result). This alerts the employer 102 if the driver 104 is not qualified to continue working if the test result is positive. The test report (containing details of the specimen analysis) can be sent securely and directly to the driver 104, being part of the driver's personal and private medical records. The system does not relay the detailed test results to the employer 102, thus maintaining confidentiality of the driver's medical information and ensuring compliance with HIPAA and ADA laws. This same process of notifying a driver 104 to obtain a screening test, selection of a test collection site 120, and subsequent testing and reporting of the results can be used by prospective employers 102 for independent drivers 104 if an employment pre-screening test is needed.

The system can also be configured to send both the test results and test reports to independent or unaffiliated drivers 104 registered with the system. This provides those drivers 104 with the full information regarding their testing and, if positive, can assist the driver 104 in obtaining appropriate treatment and/or counseling (if desired) to regain their CDL driving privileges as described below.

The partitioned communication protocols for reporting both test results and test reports only to appropriate parties/users ensures that no medical privacy laws are violated, that employers 102 are notified if a driver 104 has tested positive and must be removed from driving duties, and confirms to all parties/users that the driver 104 has completed the required testing. One or more notifications can be sent to drivers 104 and their employer 102 if they fail to schedule their test within an appropriate time frame after being selected for testing. Such notifications can serve as reminders to the driver 104, and any deadline by which a specimen must be submitted for testing can also be communicated. In this manner, employers 102 (e.g., HR and/or Safety Managers) will have timely notice on drivers 104 who have not completed their drug test and confirmation when it has been completed and reported.

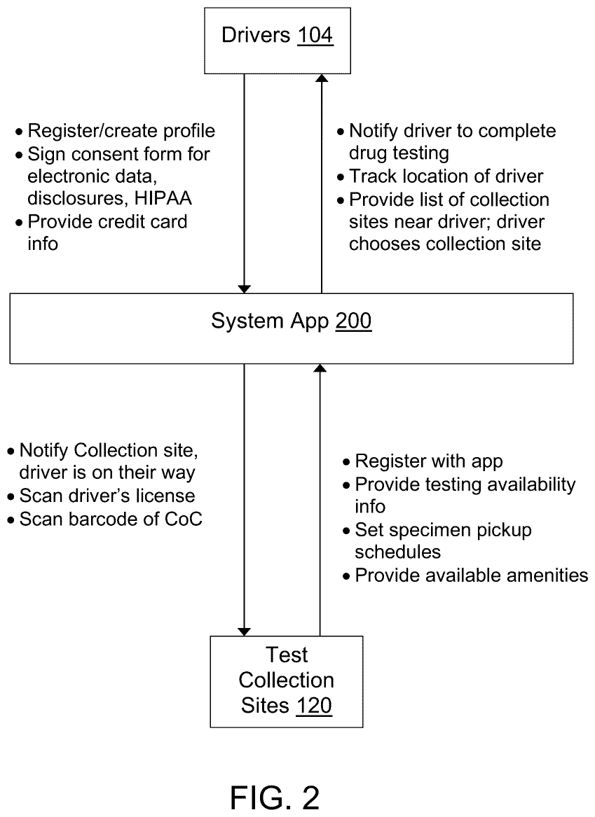

FIG. 2 is a block diagram showing some more details of communications provided by the disclosed system/platform between drivers 104, test collection sites 120, and the CA database 110 through the system app 200. These details include information provided by and to registered end users/drivers 104 using the end user system app 200, as well as information between the test collection sites 120 and the CA database 110, which can also be communicated via the system app 200. The system app 200 can be configured to provide secure communications between any end users and the CA database 110 as needed to perform various functions of the system as described herein.

For example, commercial drivers 104 can use the system app 200 to register with the disclosed system and create a user profile. The drivers 104 can also use the system app 200 to enter their credit card information (for any procedures that require payment), digitally sign/authorize a consent form for electronic data, and digitally sign any required disclosures and HIPAA agreements.

The system app 200 can also be used to inform and notify drivers 104 to schedule and complete any required drug testing. To facilitate efficient testing and reduce time off their routes, the system can further track locations of registered drivers 104 using GPS capabilities of the smartphone or other device used to run the system app 200, and provide the locations of (and optionally directions to) test collection sites 120 that are near them via the system app 200. The system app 200 can also be configured to allow a driver 104 to select a particular test collection site 120 that is convenient to them and schedule in-person collection of test samples. This information can be communicated to the CA database 110 via the system app 200.

The system app 200 can further be used to streamline the drug testing process and help to coordinate testing events by providing timely communications between the CA database 110, the test collection sites 120, and the drivers 104 being tested.

For example, the system can be configured to allow test collection sites 120 to register their location and create a site profile with the system via the system app 200, where this data can be stored in the CA database 110. Test collection sites 120 can also provide information such as their hours of operation, available amenities, and the like, to the CA database 110, where such information can then be used to help schedule testing appointments with drivers 104. Optionally, the system app 200 can also be used to help schedule pickups of test specimens by commercial carriers and track them. This can be achieved, e.g., by using conventional API functions that many commercial carriers provide to make their shipping information available and trackable in various applications.

The disclosed system can also improve the reliability and efficiency of test collection site operations in several ways. For example, the system app 200 can be configured to notify test collection sites 120 of scheduled testing appointments so they can more efficiently conduct tests and have both staff and sufficient testing materials on hand. The system app 200 can also be configured to allow test collection sites 120 to scan government issued IDs, such as driver licenses of test subjects to confirm identity. It can also be configured to scan Chain of Custody (COC) barcodes to ensure donor/specimen continuity and facilitate transportation of the specimens in accordance with 49 CFR part 40, DOT Operating Administrative Rules.

Throughout this disclosure, reference is made to a system app 200 that can perform various communication and data functions during operation of the system. Such system app 200 is used herein as a collective descriptor and may be provided as two or more separate applications that can be used by various participants in the system to perform different functions. For example, one version of the system app 200 can be provided to drivers 104 to perform various driver-related functions as described herein. Other versions of the system app 200 can be provided to different users such as driver employers 102, test collection centers 120, testing labs 130, and system administrators to perform the various particular functions associated with such users as described herein.

Further embodiments of the disclosure can also provide a system and platform for facilitating treatment and/or therapy protocols for drivers 104 who have tested positive for drugs, in order to motivate and help them to receive such help and regain their driving privileges. This feature can also simplify their locating and obtaining help from a licensed Substance Abuse Professionals (SAP) to comply with Federal regulations for commercial driving after testing positive for drug use.

For example, after a driver 104 tests positive and is restricted from driving, the system/platform, via the end user system app 200, can provide the driver 104 with a list of SAPs in close proximity to the driver's home address or any other provided location. Once the driver 104 chooses a SAP, and with the driver's consent, the drug test report can be encrypted by the CA database system 110 and sent to the Substance Abuse Professional for further evaluation. The driver 104 can then connect with the selected SAP directly, and comply with any follow-up testing and return-to-duty testing requirements as prescribed by the SAP.

SAPs may evaluate drivers 104 remotely or in person. If the SAP chooses to evaluate the driver 104 remotely, the system can provide an appropriate interface (e.g., visual teleconference connections) or direct communication information (e.g. a web link to allow the driver and SAP to teleconference, etc.) to allow the SAP to meet the criteria for conducting remote Evaluations as defined below:

Qualified substance abuse professionals (SAPs) may conduct assessments either in-person or remotely but must adhere to certain requirements when performing assessments remotely. For remote assessments, the SAP must use technology permitting real-time, two-way audio and visual interaction between the SAP and driver. This technology must be of sufficient quality so the SAP is able to gather all the visual and audible information they would normally observe during an in-person session. The technology used must also provide adequate security to protect the confidentiality of the conversation. SAPs must abide by geographic limitations applicable to their credentials for these remote assessments. SAPs who are unsure of their geographic limitations may contact the organization that provided their credentials. The system can also allow the SAP to input their geographical constraints, which can be used by the system to filter available SAPs and only present those qualified to work with the driver based on location.

The platform can also provide an encrypted web interface for SAPs to schedule all follow up testing for the driver 104. The follow up testing schedule can be hidden from drivers 104 to help avoid driver “preparation” for known upcoming test dates. The system can provide resources to the driver 104 to schedule any further tests and receive the results as described above for the randomized testing protocol, to ensure the driver 104 completes the follow up testing schedule as ordered by the SAP. The system and platform described herein can be configured to receive and store a prescribed testing schedule provided by the SAP for that driver 104, so that the driver 104 can be notified, e.g., on the day that testing is required and be provided with the test collection site location.

Results of any follow up tests or return to work tests can be reported automatically by the system to the driver's employer's HR department or Designated Employer Representative (DER). Such reporting meets Federal compliance requirements. As with the randomized testing program, only the test results (positive or negative) are reported to the HR personnel or DER, and not the full test report that contains details of the actual testing. This again ensures that the driver's private medical information is not accessible by their employer 102, thus guaranteeing compliance with HIPAA and ADA laws. Both the test results and the test report can also be uploaded to the Management Information System (MIS) to comply with government protocols. Conventional reporting systems, which are performed manually by certain employees, generally do not and cannot guarantee the direct reporting of only test results (and not full test reports) to driver employers 102, despite the existence of regulations that prohibit reporting of full test reports to employers 102.

This post-positive result feature of the disclosed system makes it a lot easier for a driver 104 to obtain services from an SAP after testing positive, and also makes it more convenient for both the driver 104 and SAP to engage in appropriate therapy and/or treatment. It also simplifies the scheduling and reporting activities of the SAP so they can focus more on the actual interactions with the driver 104.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof. It will thus be appreciated that those skilled in the art will be able to devise numerous systems, arrangements and methods which, although not explicitly shown or described herein, embody the principles of the invention and are thus within the spirit and scope of the present invention

What is claimed is:

1. A system for managing a drug testing program for commercial drivers, comprising:
a computer server comprising a memory arrangement hosting server software and a database in non-volatile memory, and provided with secure network communication capabilities;
a driver app configured to run on a personal communication device; and
a testing site app;
wherein the personal communication device comprises at least one of a smartphone or a tablet, and has location-sensing capabilities and wireless network communication capabilities;
wherein the driver app is configured to receive user input from a driver and send registration data provided by the driver to the database;
wherein the registration data comprises personal information associated with the driver and a driver license number associated with the driver;
wherein the testing site app is configured to receive user input from a testing site and send testing site information to the database;
wherein the testing site information comprises a location of the testing site and availability of the testing site to perform drug tests;
wherein the server software is configured to receive driver selection information from an entity governing the drug testing program;
wherein the server software is further configured to send a notice of testing to the personal communication device associated with a particular driver selected by the entity, and to provide a list of testing sites to the particular driver based on at least one of a location of the particular driver's location detected by the particular driver's personal communications device or a route being driven by the particular driver;
wherein the server software is configured to receive a selection of a particular testing site by the particular driver via the driver app, generate a testing appointment based on the testing site information associated with the particular testing site, and confirm the testing appointment with the particular driver and the particular testing site via the driver app and the testing site app respectively.

2. The system of claim 1, wherein:
the server software is further configured to receive drug test results from a medical review officer based on test samples provided by the particular driver to the particular testing site, store the results in the database, and send at least a portion of the drug test results to the particular driver via the driver app.

3. The system of claim 2, wherein the server software is further configured to send a further portion of the drug test results to an employer associated with the particular driver via the driver app, wherein the further portion is different from the portion of the drug test results sent to the particular driver.

4. The system of claim 1, further comprising a counselor app configured to receive user input from a certified drug counselor on a computing device associated with the drug counselor, and to send counselor registration data provided by the counselor to the database;
wherein the counselor registration data comprises contact information for the counselor, a location of the counselor, and availability data of the counselor to provide counseling services;
wherein the server software is configured to provide a list of available counselors to a positive-testing driver via the driver app based on location data associated with the positive-testing driver and on the counselor registration data; and
wherein the server software is further configured to receive a selection of a particular counselor from the list of available counselors by the positive-testing driver via the driver app, generate a counseling appointment based on the selection of the particular counselor, and confirm the counseling appointment with the positive-testing driver and the selected counselor via the driver app and the counselor app respectively.
